(19) United States
(12) Patent Application Publication
(10) Pub. No.: US 2020/0261887 A1
(43) Pub. Date: Aug. 20, 2020

(54) Improvements in Solid Phase Micro-Extraction Substrate Coatings

(71) Applicant: University of South Australia, Adelaide (AU)

(72) Inventors: Hans-Jürgen Wirth, Kallista (AU); Neeraj Verma, Mawson Lakes (AU)

(21) Appl. No.: 16/634,565

(22) PCT Filed: Jul. 25, 2018
(86) PCT No.: PCT/AU2018/000121

§ 371 Date: Jan. 27, 2020

(30) Foreign Application Priority Data
Jul. 25, 2017 (AU) 2017902914

(51) Int. Cl. B01J 20/286; C12M 1/00; B01J 20/26; B01J 20/28; B01J 20/32

(52) U.S. Cl. CPC B01J 20/286; C12M 47/04; B01J 20/264; B01J 20/28016; B01J 20/2803; B01J 20/3225; B01J 20/3212; B01J 20/3223; B01J 20/3246; B01J 20/321; B01J 20/3204

(57) ABSTRACT

A solid phase microextraction substrate is disclosed. The solid phase microextraction substrate has a sorbent coating on at least part of a surface thereof. The coating is adapted for extracting at least one analyte component from a fluid matrix. The coating includes sorbent particles in a polymeric adhesive matrix. A majority of pores in each sorbent particle in the coating do not contain substantially any of the polymeric adhesive matrices.

### PRIORITY DOCUMENT

The present application claims priority from Australian Provisional Patent Application No. 2017902914 titled “IMPROVEMENTS IN SOLID PHASE MICRO-EXTRACTION SUBSTRATE COATINGS” and filed on 25 Jul. 2017, the content of which is hereby incorporated by reference in its entirety.

### TECHNICAL FIELD

The present disclosure relates to substrates for use in solid phase micro-extraction and more particularly to coatings for solid phase micro-extraction substrates, such as solid phase micro-extraction fibres.

### BACKGROUND

Analytical techniques such as liquid-liquid extraction and solid phase extraction are routinely used for the analysis of specific components (“analytes”) present in complex mixtures. In general, such analyses involve sampling, sample preparation, separation, detection and data analysis. However, sample preparation procedures using solvents (e.g. in liquid-liquid extractions) are time consuming, labour-intensive and multi-stage operations. Solid-phase extraction cartridges or discs and microwell plates have reduced many of the limitations of classical liquid-liquid extraction methods. Nevertheless, solid-phase extraction methods are still time-consuming multi-step processes.

Solid-phase microextraction (“SPME”) techniques have overcome many of the disadvantages of liquid-liquid extraction and solid phase extraction methods. SPME integrates sampling, extraction, concentration and sample introduction into a single solvent-free step in gas chromatography and dramatically reduces solvent consumption in liquid chromatography. SPME involves the use of an SPME substrate, such as an SPME fibre coated with an extracting phase, that can be a polymer or a solid (sorbent), which extracts distinct kinds of analytes from various kinds of matrices. Analytes in the sample are directly extracted and concentrated to the SPME fibre. The method saves preparation time and disposal costs and can improve detection limits. SPME is now routinely used in combination with gas chromatography (GC), GC/mass spectrometry (GC-MS), high-performance liquid chromatography (HPLC) and HPLC/mass spectrometry (HPLC-MS). In GC and GC-MS systems, the analyte(s) is thermally desorbed from an SPME fibre, whereas in HPLC and HPLC-MS systems a desorption chamber is used for solvent desorption prior to liquid chromatographic separation. The main advantage of SPME is good analytical performance combined with simplicity and low cost.

The analytical potential of SPME for extraction of the target analytes from in-vivo systems has become exceedingly important. Biocompatible SPME fibres for liquid phase/in-vivo studies were first reported in 2009 (United States Patent Application No. 2009/0026122 A1). Fibres such as these remain in commercial use today but, in use, it is often found that these fibres suffer from relatively low binding capacities and slow adsorption/desorption kinetics and cause stress in organisms.

There is a need to develop an improved fibre based technology for SPME which can overcome or ameliorate one or more of the problems with known SPME fibres and/or provide a useful alternative to known SPME fibres.

### SUMMARY

The present disclosure arises from the inventor(s) research into coating of SPME fibres with HPLC stationary phase sorbent particles that are attached to the fibres or rods with a polymer matrix. Specifically, the present inventor(s) have found that by using pre-polymerization steps for the matrix precursor and/or blocking the pores of the sorbent particles to prevent ingress of the matrix precursor prior to polymerization it is possible to form SPME fibres displaying an increase in the binding capacity per volume unit of bed is 400% compared to the commercial market leader product. Furthermore, the elution time was reduced from a recommended 30 minutes for the commercial market leader product to about 10 seconds.

According to a first aspect, there is provided a solid phase microextraction substrate having a sorbent coating on at least part of a surface thereof, the coating being adapted for extracting at least one analyte component from a fluid matrix, the coating comprising sorbent particles in a polymeric adhesive matrix and characterised in that a majority of pores in each sorbent particle in the coating do not contain substantially any of the polymeric adhesive matrix.

According to a second aspect, there is provided a solid phase microextraction substrate having a sorbent coating on at least part of a surface thereof, the coating being adapted for extracting at least one analyte component from a fluid matrix, the coating comprising sorbent particles in a polymeric adhesive matrix and characterised in that the binding capacity of the substrate per volume unit of bed is greater than 100% compared to a commercial SPME LC fibre probe coated with 45 μm thickness proprietary polymeric material and C18 bonded porous silica sorbent particles and available from Supelco INC. as SPME LC Fibre Probe, C18; df 45 μm (Sigma-Aldrich Part. No.: 57281-U Supelco) (hereafter referred to as a “commercial C18 SPME LC Fibre Probe”).

According to a third aspect, there is provided a solid phase microextraction substrate having a sorbent coating on at least part of a surface thereof, the coating being adapted for extracting at least one analyte component from a fluid matrix, the coating comprising sorbent particles in a polymeric adhesive matrix and characterised in that the elution time for an analyte of interest from the substrate is less than 30 minutes.

According to a fourth aspect, there is provided a process for preparing a solid phase microextraction substrate having a sorbent coating on at least part of a surface thereof, the coating being adapted for extracting at least one analyte component from a fluid matrix, the process comprising:

forming a sorbent particle/adhesive precursor composition comprising a polymeric matrix adhesive precursor material and sorbent particles under conditions to substantially prevent ingress of the polymeric matrix adhesive precursor material into pores of the sorbent particles in the sorbent particle/adhesive precursor composition;

coating at least part of a substrate with the sorbent particle/adhesive precursor composition; and

polymerising the polymeric adhesive precursor material in the sorbent particle/adhesive precursor composition under conditions to form a sorbent coating comprising sorbent particles in a polymeric adhesive matrix.

According to a fifth aspect, there is provided a process for preparing a solid phase microextraction substrate having a sorbent coating on at least part of a surface thereof, the coating being adapted for extracting at least one analyte component from a fluid matrix, the process comprising:

treating sorbent particles with a pore filling agent under conditions to block substantially all of the pores of the particles to form blocked pore sorbent particles;

combining the blocked pore sorbent particles and a polymeric adhesive matrix precursor material to form a sorbent particle/adhesive precursor composition;

coating at least part of a substrate with the particle/adhesive precursor composition;

polymerising the polymeric adhesive precursor material in the particle/adhesive precursor composition under conditions to form a coating on the substrate comprising blocked pore sorbent particles in a polymeric adhesive matrix; and

treating the blocked pore sorbent particles in the polymeric adhesive matrix to substantially remove the pore filling agent from the pores thereof to form the sorbent coating comprising sorbent particles in a polymeric adhesive matrix.

According to an sixth aspect, there is provided a process for preparing a solid phase microextraction substrate having a sorbent coating on at least part of a surface thereof, the coating comprising sorbent particles in a polymeric adhesive matrix and being adapted for extracting at least one analyte component from a fluid matrix, the process comprising:

combining a polymeric adhesive matrix precursor material and sorbent particles to form a sorbent particle/adhesive precursor composition comprising adhesive matrix precursor polymers or pre-polymers having a molecular size that is greater than a maximum pore size of the sorbent particles;

coating at least part of a substrate with the sorbent particle/adhesive precursor composition;

polymerising the polymeric adhesive precursor material in the sorbent particle/adhesive precursor composition under conditions to form a sorbent coating comprising sorbent particles in a polymeric adhesive matrix.

The processes of the fourth, fifth and sixth aspects can be used to prepare a solid phase microextraction substrate in which a majority of pores in each sorbent particle in the coating do not contain any of the polymeric adhesive matrix.

According to a seventh aspect, there is provided a solid phase microextraction substrate prepared by the process of any one of the fourth, fifth or sixth aspects.

According to an eighth aspect, there is provided a use of a solid phase microextraction substrate of any one of the first, second or third aspects in a solid phase microextraction process.

### BRIEF DESCRIPTION OF DRAWINGS

Embodiments of the present disclosure will be discussed with reference to the accompanying drawings wherein:

FIG. 1 shows a schematic representation of the effect of molecular size on polymerisation (curing) of a polymeric adhesive matrix precursor material in the presence of sorbent particle pores exposed to a polymeric adhesive matrix precursor material having a small molecular size (left) and one having a larger molecular size (right);

FIG. 2 shows a plot of withdrawing speed vs coating thickness;

FIG. 3 shows a plot of the amount of particles (g) vs coating thickness (microns);

FIG. 4 shows a plot of the amount of particles (g) vs coating thickness (microns);

FIG. 5 shows a plot of concentration of 3-nitroaniline in feed solution vs of concentration of 3-nitroaniline in an eluted sample;

FIG. 6 shows a plot of measured concentration of 3-nitroaniline (c*) vs the amount of bound 3-nitroaniline per gram (q*);

FIG. 7 shows a plot of measured concentration of 3-nitroaniline (c*) vs the amount of bound 3-nitroaniline per gram/measured concentration of 3-nitroaniline (q*/c*);

FIG. 8 shows a plot of the Renkin equation;

FIG. 9 shows a schematic representation of the fabrication of a silica coated fibre;

FIG. 10 shows plots of (A) nitrogen adsorption-desorption isotherms; and (B) pore-diameter distribution on different days of pre-polymerization;

FIG. 11 shows plots of binding capacity of the coated fibre at different stages of the matrix pre-polymerisation (1, 7 and 14 days) for the β-blockers, propranolol (A) and metoprolol (B);

FIG. 12 shows a schematic representation of the dip coating protocol;

FIG. 13 shows a plot showing the effect of different solvents on the coating after the exposure to the different solvents (n=3);

FIG. 14 shows a plot of number of coatings v coating thickness for multiple coating measurements on the fibres;

FIG. 15 shows a plot of number of fibres v coating thickness for the coating process on different fibres using an in-house build coating motor;

FIG. 16 shows SEM micrographs of a pre-polymerized coated fibre at different magnifications;

FIG. 17 shows survey XPS spectra of: (A) control glass; (B) glass treated with Piranha solution; and (C) glass treated with (3-glycidyloxypropyl) trimethoxysilane;

FIG. 18 shows the cell viability results of primary human foreskin fibroblast (HFF) cells exposed with “SIFT” at three exposure times (Day-1, 2 and 7). The cell viability value was determined by resazurin assay, and results are expressed as % cell viability after measuring the fluorescence signal with 530 nm excitation and 590 nm emission using 96 well plate reader. Data are shown as mean±SD (n=3). All the group shows non-significance results (<0.05);

FIG. 19 shows survey XPS spectra of control stainless steel (lower trace) and acrylic acid plasma polymer treated stainless steel fibres (upper trace);

FIG. 20 shows the peak fitting C1s core level of plasma polymer of acrylic acid; and

FIG. 21 shows SEM micrographs of a coated stainless steel fibres at different magnifications.

### DESCRIPTION OF EMBODIMENTS

Provided herein is a solid phase microextraction substrate having a sorbent coating on at least part of a surface thereof. The coating is adapted for extracting at least one analyte component from a fluid matrix. The coating comprises sorbent particles in a polymeric adhesive matrix. A majority of pores in each sorbent particle in the coating do not contain substantially any of the polymeric adhesive matrix.

The solid phase microextraction substrate described herein may provide one or more advantages. The lack of substantially any of the polymeric adhesive matrix in the pores in each sorbent particle causes a significant increase in the binding capacity of the coated solid phase microextraction substrate. The lack of substantially any of the polymeric adhesive matrix in the pores in each sorbent particle causes a dramatic reduction in the mass transfer restriction, thus leading to faster adsorption and desorption times. Faster on/off kinetics allow these substrates to be used in high throughput experiments. Faster elution in direct detection experiments (e.g. in an Open Port Probe) generates sharper elution peaks and therefore better sensitivities.

The solid phase microextraction substrate can be any substrate or device that is suitable for use in a SPME system. Suitable substrates include wires, rods or fibres (collectively referred to herein as “fibres”), as is known in the art. Suitable fibre materials include metal, glass, silica, carbon, ceramic or plastic. Suitable metals include Nitinol (Ni—Ti), stainless steel, titanium, and copper. Suitable plastics include polyether ether ketone (PEEK) or polyamide (nylon) for example. For some applications, such as in vivo applications, the fibre material is preferably biocompatible and, therefore, amenable for use in a biological matrix. The diameter of the fibre can be of millimetre to nanometre dimensions. For example, the outer diameter of the fibre can be between about 0.1 millimetres and about 6 millimetres. For some applications, the outer diameter of the fibre is about 0.25 millimetres. For other applications, such as for use in an “Open Port Probe” the fibre will have a larger outer diameter, such as about 5 mm. The geometry of the solid phase microextraction substrate is not limited to fibres and may have different geometrical formats such as those for use in planar SPME (PSPME) or membrane SPME (MSPME).

The sorbent coating comprises sorbent particles in a polymeric adhesive matrix and is surface bonded on at least part of a surface of the substrate. The thickness of the sorbent coating may be from about 3 microns to about 1000 microns, for example from about 3 microns to about 350 microns. For example, the thickness of the sorbent coating may be 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200 microns. In certain embodiments, the thickness of the sorbent coating is 30 microns. In certain embodiments, the thickness of the sorbent coating is 45 microns. In certain embodiments, the thickness of the sorbent coating is 100 microns. In certain embodiments, the coating thickness is from about 20 microns to about 50 microns, leading to an increase in diameter of the fibre of from about 40 microns to about 70 microns. The thickness of the coating is determined, at least in part by the nature of the sorbent particles used, the composition of the polymeric adhesive matrix, the outer diameter of the SPME fibre and the speed at which the SPME substrate to be coated is withdrawn from a sorbent particle/adhesive matrix precursor composition.

Optionally, the substrate may be surface treated prior to it being coated with the sorbent particle/adhesive matrix precursor composition. The surface treatment may promote polymer adhesion. For example, silica substrates may be hydrolysed to expose the hydroxyl groups on the surface of the substrate. Silica substrates can be hydrolysed using techniques known in the art such as chemical etching with strong acid (e.g. piranha solution) or alkaline solution, hydrothermal treatment or by exposure to plasma. Optionally, silica substrates may be silanized by treating the substrates with a silane. A wide range of silanes are available commercially (see for example www.gelest.com/product-lines/silanes/?pl_page=1&perpage=100) and many of the known silanes can be used. For example, the silane may be aminopropyltriethoxysilane, 3-(trimethoxysilyl) propyl methacrylate or (3-glycidyloxypropyl) trimethoxysilane.

The surface treatment may be a plasma treatment using a suitable monomer to form a plasma polymer intermediate coating. A range of monomers are known for use in plasma surface modification of metal surfaces (for example) and any of these may be suitable for the plasma treatment. In certain embodiments, the monomer is an alkene monomer, a carboxylate monomer or a combination thereof. By way of example, the alkene monomer may be acrylic acid. By way of example, the carboxylate monomer may be propanoic acid.

The sorbent coating is adapted for extracting at least one analyte component from a fluid matrix. The matrix can be an environmental sample, a food sample, a biological fluid, tissue, organ or cell. The biological fluid can be whole blood, plasma, serum, urine, cerebrospinal fluid, saliva or peritoneal fluid. The analyte can be any compound whose presence at a location is indicative of one with biological, environmental, food, pharmaceutical, bio-analytical, clinical, forensic, toxicological, national security, public health, and/or safety implications. For example, the solid phase microextraction substrate described herein can be used for in vitro analysis of biological analytes as well as for in vivo analysis of biological analytes in a living animal. Alternatively, or in addition, the solid phase microextraction substrate described herein can be used for analysis of small molecules such as drugs or biomarkers.

It will be understood by those skilled in the art that the sorbent particles can be chosen to analyse specific target analytes. The sorbent particles can be particles of any sorbent material that is able to bind to one or more target analytes of interest. Sorbents that are commonly used in liquid chromatography, such as derivatized silica particles, can be used. For example, the sorbent can be C-18/silica particles, RP-amide/silica particles, HS-F5/silica particles, normal-phase silica particles, C-1/silica particles, C-4/silica particles, C-6/silica particles, C-8/silica particles, C-30/silica particles, phenyl/silica particles, cyano/silica particles, ionic liquid/silica particles, molecular imprinted polymer particles, carboxen particles, styrene/divinylbenzene particles, diol/silica particles, particles with immobilised bio-specific ligands such as antibodies or mixtures thereof. The sorbent particles can be about 1 μm to about 50 μm particles, such as 3 μm, 5 μm or 10 μm. The sorbent particles can have a surface area of about 20 m²/g to about 800 m²/g. The sorbent particles can have pore sizes from about 10 Angstroms to about 2000 Angstroms, such as about 100 Angstroms, about 120 Angstroms, about 200 Angstroms or about 300 Angstroms. Larger pore size sorbent particles may be useful for immobilisation of proteins such as antibodies.

The polymeric adhesive matrix is a polymeric adhesive material that adheres to the surface of the substrate and to the sorbent particles. The polymeric adhesive matrix is formed from an adhesive matrix precursor composition. In certain embodiments, the polymeric adhesive matrix is a polyamine epoxy. The epoxy component of the polyamine epoxy may be an epoxy resin having at least 2 epoxy groups. Examples of epoxies that can be used include epoxy polyethers of polyhydric phenols obtained by reacting a polyhydric phenol with a halogen containing epoxide in an alkaline medium. Polyhydric phenols that can be used for this purpose include, among others, resorcinol, catechol, hydroquinone, methyl resorcinol, or polynuclear phenols, such as 2,2-bis (hydroxyphenyl) propane (bisphenol A), 2,2-bis(4-hydroxyphenol) butane, 4,4′-dihydroxybenzophenone, bis(4 hydroxyphenyl) ethane, and 2,2-bis(4-hydroxy-phenol) pentane. The halogen-containing epoxides may be 3-chloro-1,2-epoxybutane, 3-bromo-1,3-epoxyhexane, 3-chloro-1,2-epoxyoctane, and the like.

The polyamine component of the polyamine epoxy is a curing agent that may be an aliphatic polyamine or a cycloaliphatic polyamine Useful polyamines contain from about 2 to about 6 amine nitrogen atoms per molecule and from 2 to about 20 carbon atoms. Examples of suitable amines are the alkylene polyamines, ethylene diamine, 1,2-propylene diamine, 1,3-propylene diamine, 1,2-butylene diamine, 1,3-butylene diamine, 1,4-butylene diamine, 1,5-pentalene diamine, 1,6-hexylene diamine, methane diamine, 1,4-diaminocyclohexane, diethylene triamine, triethylene tetramine, tetraethylene pentamine, pentaethylene hexamine, dipropylene triamine, tributylene tetramine, hexamethylene diamine, dihexamethylene triamine and the like. Mixtures of polyamines can also be used.

Optionally, solid phase microextraction substrates for in vivo analysis may be coated with a biocompatible outer coating, such as polyacrylonitrile (PAN), polyethylene glycol, polypyrrole, derivatised cellulose, polysulfone, polyamide, or polycarbohydrates such as dextran or chitin. Examples of the biocompatible coating that can be used include: a PAN/C-18 coating, a PAN/RP-amide coating, a polyethylene glycol/HS-F5 coating, a derivatised cellulose/C-18 coating, a polypyrrole/C-30 coating, a polysulfone/phenyl coating and polyamide/cyano coating.

Optionally, solid phase microextraction substrates for in vivo analysis may be coated with a hydrophilic outer coating. The hydrophilic coating can be used to suppress binding of proteins and other large molecules to the coating. Dextran is a suitable hydrophilic material.

Advantageously, a majority of pores in each sorbent particle in the coating do not contain substantially any of the polymeric adhesive matrix. As used herein, that term means greater than 50% of the pores in each sorbent particle in the coating do not contain any of the polymeric adhesive matrix, greater than 55% of the pores in each sorbent particle in the coating do not contain any of the polymeric adhesive matrix, greater than 60% of the pores in each sorbent particle in the coating do not contain any of the polymeric adhesive matrix, greater than 65% of the pores in each sorbent particle in the coating do not contain any of the polymeric adhesive matrix, greater than 70% of the pores in each sorbent particle in the coating do not contain any of the polymeric adhesive matrix, greater than 75% of the pores in each sorbent particle in the coating do not contain any of the polymeric adhesive matrix, greater than 80% of the pores in each sorbent particle in the coating do not contain any of the polymeric adhesive matrix, greater than 85% of the pores in each sorbent particle in the coating do not contain any of the polymeric adhesive matrix, greater than 90% of the pores in each sorbent particle in the coating do not contain any of the polymeric adhesive matrix or greater than 95% of the pores in each sorbent particle in the coating do not contain any of the polymeric adhesive matrix.

Solid phase microextraction substrates can be formed so that a majority of pores in each sorbent particle in the coating do not contain substantially any of the polymeric adhesive matrix by preventing or minimising ingress of the polymeric matrix adhesive precursor material into pores of the sorbent particles in the sorbent prior to polymerisation or curing of the polymeric matrix adhesive precursor material. Thus provided herein is a process for preparing a solid phase microextraction substrate having a sorbent coating on at least part of a surface thereof. The coating is adapted for extracting at least one analyte component from a fluid matrix. The process comprises forming a sorbent particle/adhesive precursor composition comprising a polymeric matrix adhesive precursor material and sorbent particles under conditions to substantially prevent ingress of the polymeric matrix adhesive precursor material into pores of the sorbent particles in the sorbent particle/adhesive precursor composition. At least part of a substrate is then coated with the sorbent particle/adhesive precursor composition and the polymeric adhesive precursor material in the sorbent particle/adhesive precursor composition is polymerised under conditions to form a sorbent coating comprising sorbent particles in a polymeric adhesive matrix.

Our results show that preventing ingress of the polymeric matrix adhesive precursor material into the pores of the sorbent particles during the coating causes a significant increase in the binding capacity of the coated solid phase microextraction substrate and causes a dramatic reduction in the mass transfer restriction thus leading to faster adsorption and desorption times relative to known SPME fibres.

In certain embodiments, the “conditions to substantially prevent ingress of the polymeric matrix adhesive precursor material into pores of the sorbent particles” comprise blocking the pores of the sorbent particles with a pore blocking agent. Thus, provided herein a process for preparing a solid phase microextraction substrate having a sorbent coating on at least part of a surface thereof. The coating is adapted for extracting at least one analyte component from a fluid matrix. The process comprises treating sorbent particles with a pore filling agent under conditions to block substantially all the pores of the particles to form blocked pore sorbent particles. The blocked pore sorbent particles and a polymeric adhesive matrix precursor material are then combined to form a sorbent particle/adhesive precursor composition. At least part of a substrate is then coated with the particle/adhesive precursor composition and the polymeric adhesive precursor material in the particle/adhesive precursor composition is cured under conditions to form a coating on the substrate comprising blocked pore sorbent particles in a polymeric adhesive matrix. The blocked pore sorbent particles in the polymeric adhesive matrix are then treated to substantially remove the pore filling agent from the pores thereof to form the sorbent coating comprising sorbent particles in a polymeric adhesive matrix.

The pore filling agent can be any material that is able to fill the pores of the sorbent particles and remain in the pores during the polymerisation step for the polymeric adhesive precursor material. Suitable pore filling agents include hexadecanol. Hexadecanol has a melting point of 49.3 degrees C. and is, therefore solid at room temperature and liquid at temperatures above about 50 degrees C. Thus, the sorbent particles can be mixed with liquid hexadecanol and then removed from the hexadecanol and allowed to cool to less than 50 degrees C. at which point the hexadecanol solidifies in the pores. After coating onto the substrate, the sorbent particles can be heated to over 50 degrees C. and the liquid hexadecanol can be removed from the pores of the sorbent particles under vacuum. Other agents having similar melting point and/or hydrophobicity (log P=6.14) to hexadecanol could also be used. Examples of other agents that could be used include paraffin waxes and other long chain alcohols.

The pore filling agent may be removed from the pores of the sorbent particles using any suitable technique, including heating, exposure to vacuum, solvation with a suitable solvent, chemical manipulation of the pore filling agent in the pores, chemical or physical degradation of the pore filling agent in the pores, and the like.

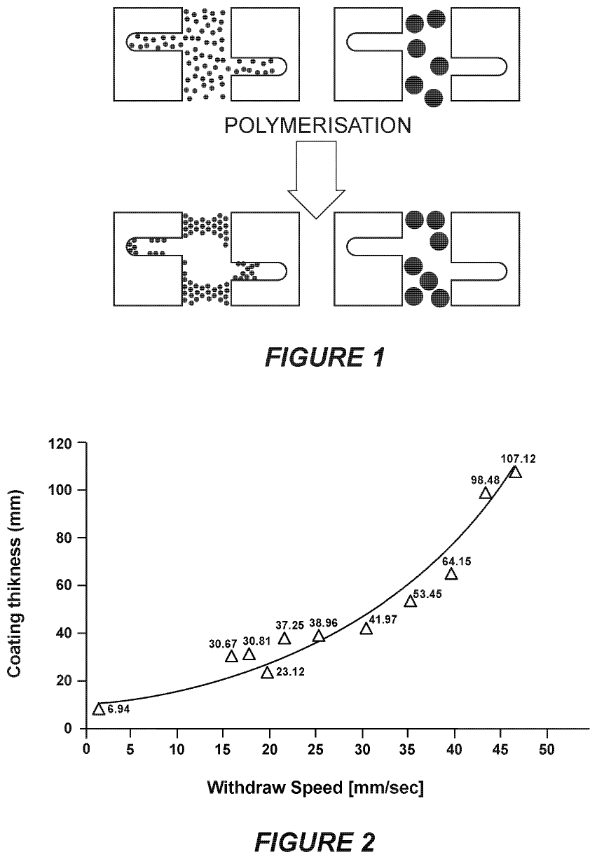

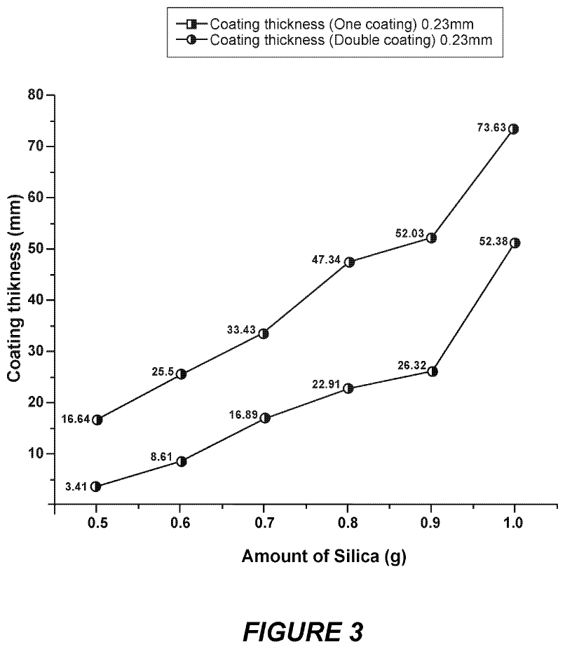

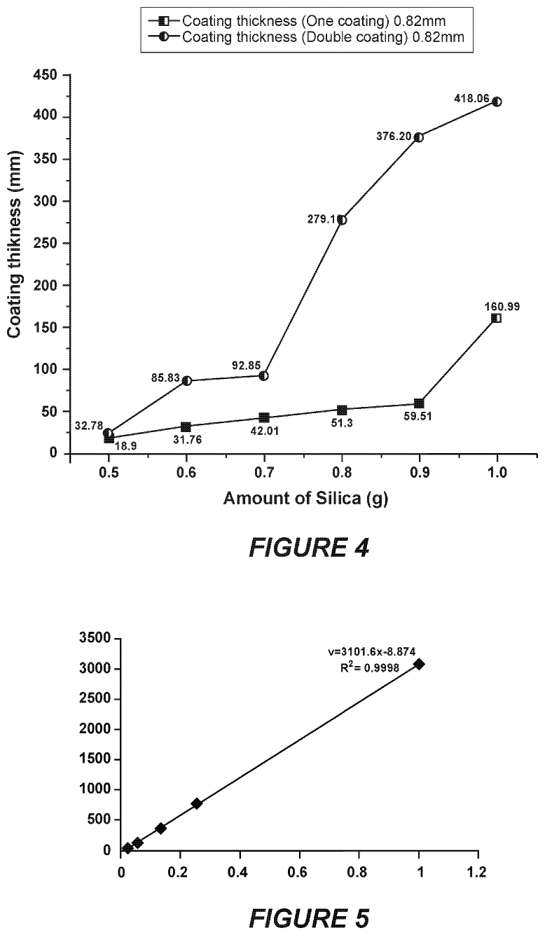

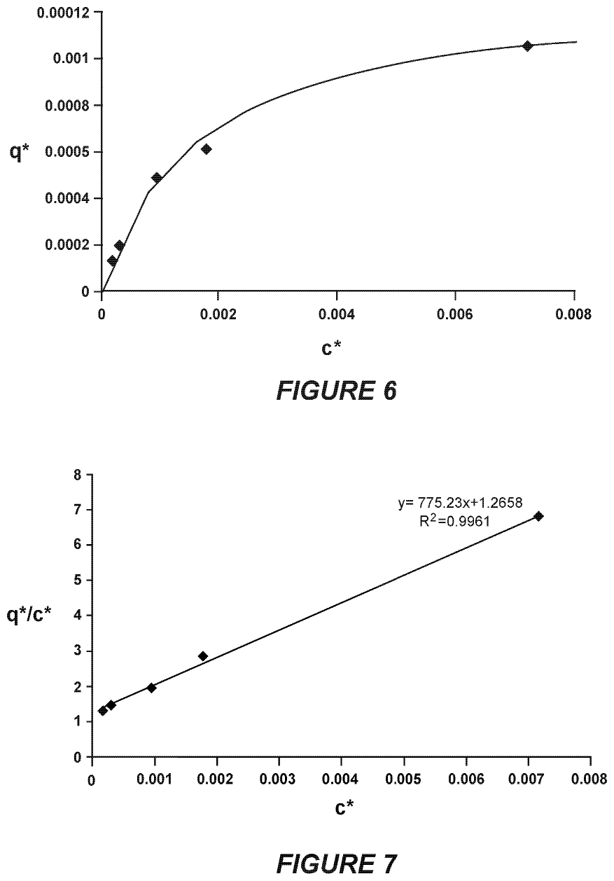

In certain other embodiments, the “conditions to substantially prevent ingress of the polymeric matrix adhesive precursor material into pores of the sorbent particles” comprise contacting the sorbent particles with adhesive matrix precursor polymers or pre-polymers that have a molecular size that is greater than a maximum pore size of the sorbent particles. This is shown schematically in FIG. 1. In this way, the adhesive matrix precursor polymers or pre-polymers are not able to enter the pores of the sorbent particles prior to or after curing. Thus, provided herein is a process for preparing a solid phase microextraction substrate having a sorbent coating on at least part of a surface thereof. The coating comprises sorbent particles in a polymeric adhesive matrix that is adapted for extracting at least one analyte component from a fluid matrix. The process comprises combining a polymeric adhesive matrix precursor material and sorbent particles to form a sorbent particle/adhesive precursor composition comprising adhesive matrix precursor polymers or pre-polymers having a molecular size that is greater than a maximum pore size of the sorbent particles. At least part of a surface of a substrate is then coated with the sorbent particle/adhesive precursor composition after which it is polymerised under conditions to form a sorbent coating comprising sorbent particles in a polymeric adhesive matrix.

The adhesive matrix precursor polymers or pre-polymers can be formed by starting polymerisation of the polymeric adhesive material and adding the sorbent particles to the reaction after polymerisation has started but before it is finished. If required, the state of polymerisation of the adhesive matrix precursor material can be determined using known techniques, including by measuring the viscosity of the reaction mixture and adding the sorbent particles at a time when a predetermined viscosity is reached. The skilled person will appreciate that the viscosity of the reaction mixture increases as the level of polymerisation increases.

We found that we were able to increase the binding capacity per volume unit of bed by 400% compared to the market leader product. Thus, also provided herein is a solid phase microextraction substrate having a sorbent coating on at least part of a surface thereof, the coating being adapted for extracting at least one analyte component from a fluid matrix, the coating comprising SPME particles in a polymeric adhesive matrix and characterised in that the binding capacity of the substrate per volume unit of bed is greater than 100% compared to the commercial C18 SPME LC Fibre Probe.

The binding capacity of the substrate per volume unit of bed may be greater than 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390% or 400% compared to the commercial C18 SPME LC Fibre Probe.

In certain specific embodiments, the binding capacity of the substrate per volume unit of bed is 400% compared to the commercial C18 SPME LC Fibre Probe.

The desorption time for eluting an analyte from the commercial C18 SPME LC Fibre Probe is recommended to be 30 minutes. In contrast, we found that the elution time for an analyte (i.e. 3-nitoraniline) from a solid phase microextraction substrate described herein was 10 seconds or less. Thus, also provided herein is a solid phase microextraction substrate having a sorbent coating on at least part of a surface thereof, the coating being adapted for extracting at least one analyte component from a fluid matrix, the coating comprising SPME particles in a polymeric adhesive matrix and characterised in that the elution time for an analyte of interest from the substrate is less than 30 minutes.

The elution time for an analyte of interest from the substrate may be less than 29 minutes, 28 minutes, 27 minutes, 26 minutes, 25 minutes, 24 minutes, 23 minutes, 22 minutes, 21 minutes, 20 minutes, 19 minutes, 18 minutes, 17 minutes, 16 minutes, 15 minutes, 14 minutes, 13 minutes, 12 minutes, 11 minutes, 10 minutes, 9 minutes, 8 minutes, 7 minutes, 6 minutes, 5 minutes, 4 minutes, 3 minutes, 2 minutes, 1 minute, 59 seconds, 58 seconds, 57 seconds, 56 seconds, 55 seconds, 54 seconds, 53 seconds, 52 seconds, 51 seconds, 50 seconds, 49 seconds, 48 seconds, 47 seconds, 46 seconds, 45 seconds, 44 seconds, 43 seconds, 42 seconds, 41 seconds, 40 seconds, 39 seconds, 38 seconds, 37 seconds, 36 seconds, 35 seconds, 34 seconds, 33 seconds, 32 seconds, 31 seconds, 30 seconds, 29 seconds, 28 seconds, 27 seconds, 26 seconds, 25 seconds, 24 seconds, 23 seconds, 22 seconds, 21 seconds, 20 seconds, 19 seconds, 18 seconds, 17 seconds, 16 seconds, 15 seconds, 14 seconds, 13 seconds, 12 seconds, 51 seconds, 10 seconds, 9 seconds, 8 seconds, 7 seconds, 5 seconds, 4 seconds, 3 seconds, 2 seconds or 1 second.

One or more embodiments of the present disclosure may provide one or more the following advantages:
Keeping the pore system of the particles open during the coating causes a significant increase in the binding capacity of the coated device;
Keeping the pore system of the particles open during the coating also causes a dramatic reduction in the mass transfer restriction thus leading to faster adsorption and desorption times;
Faster on/off kinetics allow these fibres to be used in high throughput experiments;
Faster elution in direct detection experiments (like the Open Port Probe) generate sharper elution peaks and therefore better sensitivities;
The fibres do not swell in water or solvents; and
The fibre coating is durable and reproducible.

### EXAMPLES

### Example 1

### Preparation of C18 Coated SPME Fibre Using a “Blocked Pore” Method

Hexadecanol was dissolved in chloroform on a water bath at a ratio of 2:1. 1 ml of this solution was added to 1 g of C18 silica in a closed vial. The specific pore volume of the C18 silica used was 1 ml/g. The silica was agitated to allow for an even distribution of the solution in the particles under the assumption that capillary forces would draw the solution into the pores. The silica particles were mixed vigorously at 50° C. for 5 minutes. Over this time period, the initial clumpy silica became powdery. The vial was then opened and the particles were kept at room temperature to let chloroform evaporate from the mixture for 24 hours. The process was then repeated using another 333 μl of the solution.

The hexadecanol modified C18 silica particles were then suspended in a polymeric composite formulation comprising poly(2-hydroxyethyl methacrylate-ethylene dimethacrylate) formed from 2-hydroxyethyl methacrylate (HEMA), ethylene dimethacrylate (EDMA) using a phenylbis(2,4,6-trimethylbenzoyl)-phosphine oxide (BAPO) initiator. A metal or glass SPME fibre was dipped into the slurry and withdrawn to form a coating which was then cured. The pre-filled hexadecanol was then washed out in the organic phase. The coating thickness was dependent on the amount of silica, the polymer composition, the fibre OD, and the withdrawal speed.

### Example 2

### Preparation of C18 Coated SPME Fibre Using a “Prepolymerisation” Method

A 3M-Scotch-weld DP240 epoxy adhesive was dissolved in chloroform at a concentration of 10 weight % and the polymerisation time increased from 20 min to about 2 weeks. The polymerisation time is dependent on the concentration and the storage temperature. Higher concentrations and higher temperatures decrease the polymerisation time. During the polymerisation the viscosity of the solution constantly increases. There is a window of several days where the solution has polymerised enough that the polymer precursors are large enough not to penetrate the pores and where the solution is still liquid enough to provide a uniform coating. Prior to coating, a slurry of 0.4 g C18 silica (0.1 g-1 g range is possible) and 1 ml of adhesive solution was made. The desired coating thickness was regulated by the dipping speed. As the viscosity of the slurry increased the draw speed of the substrate from the slurry had to decrease.

### Example 3

### Performance of C18 Coated SPME Fibres

Binding capacities were measured with an aqueous solution of 3-nitroaniline. The commercial fibre was treated according to the recommendations from the supplier.

The SPME fibre of the present disclosure was pre-treated with methanol for 30 seconds followed by water for 30 sec. Adsorption was performed in 60 sec and elution was performed by trickling 1 ml of methanol along with the fibre within 20 seconds. 10 μl of the elution solution was injected onto a 250×4.6 mm HPLC column for quantitation.

### The Langmuir Adsorption Isotherm

One can look at the binding of an analyte to, for example, a C18 particle as a reversible reaction. For every x analyte molecules bound to the particle (q*) there will be y molecules left in solution (c*). The proportion between x and y is dependent on the affinity between the analyte and the C18 surface. This proportion is the affinity constant (Kₐₛₛ).

The surface area of the C18 particle is limited and therefore there are a limited number of analyte molecules this surface can accommodate. This is the maximum binding capacity (qₘ).

The relationship between all the above factors is given below (Langmuir, I. (1918) J. Am. Chem. Soc., 40, 1361-1403):

$${q}^{*} = \frac{{q}_{m} \cdot {K}_{ass} \cdot {c}^{*}}{1 + {K}_{ass} \cdot {c}^{*}}$$

If one measures the amount bound (q*) for a number of different concentrations (c*) the data can be fitted to the Langmuir equation with qₘ and Kₐₛₛ as fitting parameters.

To compare two different sorbents with the same chemistry (e.g. C18) one has to record only one concentration (c*). Provided the chemistry is the same, then Kₐₛₛ has to be the same. If q* for sorbent A is twice as high as for sorbent B then qₘ for sorbent A has to be twice as high a qₘ for sorbent B.

### Experimental

For comparative binding studies a solution 15 mg of 3-nitroaniline in 100 ml of water was prepared. The coated fibre was treated with methanol for 1 minute followed by a rinse with water for one minute. The coated fibre was then submerged in 10 ml of the 3-nitroaniline solution and the solution was stirred for 5 min to reach equilibrium. The fibre was rinsed with water and the bound 3-nitroaniline was eluted with methanol. 10 μl of the eluent was injected onto a 250 mm×4.6 mm ID C18 column. The concentration of bound 3-nitroaniline was quantified using the peak area at 232 nm wavelength.

### Restricted Pore Diffusion

Chromatographic particles are characterized by their physical properties (particle size, pore size, pore volume etc.) and their chemical properties (surface chemistry, ligand density, end-capping, non-specific binding etc.). These particles are usually packed in a bed or immobilized onto a surface and thus do not move and form a stationary phase. The sample and elution solution is a liquid which is transported across the stationary phase as a mobile phase. The surface chemistry rules the interaction between the sample and the stationary phase. Most particles used for SPE or chromatography are porous with pore sizes reaching from 5 nm (50 Å) to 400 nm (4000 Å). Pore volumes for porous silica particles are fairly constant at around 1 ml/g. The vast majority of the interactive surface lies within the pore system, with small pores having a much larger surface area (120 Å pore particles have 350 m² surface area) than large pore size particles (1000 Å pore particles have 30 m² surface area).

When liquid flows around a particle the solvent inside the pores is stationary. In order to interact with the particle surface the analyte molecule has to penetrate the pore system and the only way it can do this is by diffusion.

In a free solution the diffusion of a molecule is described by the Einstein Stokes Equation:

$${D}_{f} = \frac{{k}_{B} T}{6 π μ R}$$

where: D_{f}=Diffusion rate constant in free solution; k_{B}=Boltzmann constant; μ=viscosity; R=hydrodynamic radius of the molecule; and T=temperature.

Inside a pore, the diffusion becomes more complex with two extreme positions:
If the pore is infinitely wide then the diffusion will be equal to the Einstein-Stokes diffusion; and
If the analyte molecule is as big or bigger than the pore diameter then the diffusion will be zero.

As the pore size becomes smaller there will be an increased chance that the molecule interacts with the wall of the pore and thus slowing the diffusion down. In 1954 E. M. Renkin developed an empirical equation to describe the restricted pore diffusion in a cylindrical pore:

$${D}_{p} = {{D}_{f} ( 1 - \frac{{r}_{s}}{{r}_{p}} )}^{2} [ 1 - 2.104 ( \frac{{r}_{s}}{{r}_{p}} ) + 2.09 {( \frac{{r}_{s}}{{r}_{p}} )}^{3} - 0.95 {( \frac{{r}_{s}}{{r}_{p}} )}^{5} ]$$

where: Dₚ=Pore diffusion rate; D_{f}=Free diffusion rate; rₛ=radius of the solute molecule; and rₚ=radius of the pore.

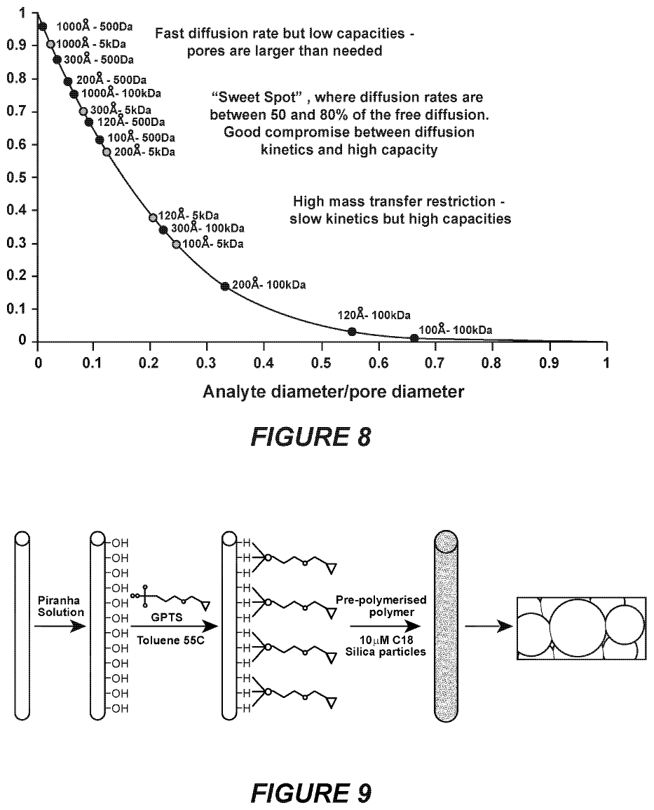

When plotted the Renkin equation is as shown in FIG. 8.

Any obstruction in a pore will slow the diffusion right down.

The Renkin model assumes cylindrical pores. When embedding porous particles in a polymeric matrix it is possible that only the pore entrance is getting obstructed while the majority of the pore volume remains open. These types of pores are commonly referred to as ink-bottle type pores. The diffusion in and out of the pore system is governed by the opening of the pore.

### Example 4

### Preparation and Performance of Coated Silica Fibres

Reagents and Materials

Polyimide coated silica fibres (357.7 μm) were obtained from Polymicro Technologies (Phoenix, Ariz.). (3-Glycidyloxypropyl) trimethoxysilane (98%), toluene, sulfuric acid (99%), hydrogen peroxide (30%), methanol, chloroform, diclofenac, metoprolol tartrate, propranolol hydrochloride, and resazurin sodium were purchased from Sigma Aldrich. Sulfuric acid (Merck Millipore), hydrogen peroxide (30%) and potassium hydroxide flakes were purchased from Chem-supply, SA-AUS. C-18 Bio-SPME fibres were purchased from Supelco (Bellefonte, Pa.). 3M-Scotch-weld DP240 epoxy adhesive was purchased from 3M, Maplewood Minn., USA. C18 silica particles were purchased from the Osaka Soda Co. Ltd. Japan. Dulbecco's Modified Eagle's Medium (DMEM), dimethyl sulphoxide (DMSO), phosphate buffered saline (PBS), lipopolysaccharide (LPS), and tris buffer were purchased from Sigma Chemical Co. Ltd. (St. Louis, Mo., USA). Fetal Bovine Serum (FBS) and antibiotic-antimycotic solution (10,000 U/ml penicillin, 10 mg/ml streptomycin sulfate) was purchased from Gibco, Invitrogen Co. (Grand Island, N.Y., USA). Cell culture plates (nunc) were obtained from ThermoFisher Scientific (Roskilde, Denmark).

Instrumentation

Chromatographic experiments were carried out using an agilent 1260 infinity liquid chromatograph (LC) with 6130 Quad mass spectrometer (MS). An analytical column ZORBAX Eclipse Plus C-18, 4.6 mm*150 mm, 3.5 μm particles with a guard column (Agilent Eclipse XDB, C-18, 4.6×12.5 mm) was used. A Zeiss Merlin scanning electron microscope (SEM) was used at 2 kv to record the images. The textural property of the coating material was analyzed using the nitrogen (N₂) sorption analyzer (micrometrics ASAP 2420). X-ray photoelectron spectroscopy (XPS) spectra of the fibre surface were recorded using a SPECS SAGE XPS system with a phoibos 150 hemispherical analyzer. The fibres were coated by dip coating using an in-house built coating motor.

Pre-Treatment of the Fibre and Hydroxylation

Polyamide-coated silica capillaries (357.5 μm OD and 49.8 μm ID) were cut into 10 cm length. The polyamide layer was removed for a length of 5 cm using a butane flame. The silica capillaries were then vortexed in methanol for 10 minutes to remove traces of burn polyamide and 5 min in water followed by nitrogen drying. The fibres were checked under a microscope to make sure no polyamide remained on the surface. The surface of the fibres was hydroxylated using piranha solution (3:1 Conc. H₂SO₄ and H₂O₂) for 90 minutes at room temperature (22° C.) to remove the contaminants from the surface and expose the hydroxyl group to the surface of the fibres. Using ultrasonication the acid treated fibres were cleaned in water, ethanol and acetone for 5 minutes. Each fibre was cleaned by ultrasonication and then the fibres were subjected to further treatment. Fibres were dried under nitrogen.

Silanization

After hydroxylation with piranha solution, different surface treatments were tested, namely: 3-aminopropyltriethoxysilane (APTES), 3-(trimethoxysilyl) propyl methacrylate; and (3-glycidyloxypropyl) trimethoxysilane (GPTS).

Using GPTS we observed a robust binding of the polymer with silica particles on the fibre. The protocol of the GPTS treatment was performed by treating the fibres with the 20% (v/v), GPTS/toluene at 55° C. for 48 hours. To remove the weakly bound silane compounds fibres were sonicated in toluene and methylene chloride for 5 minute each respectively. Finally, fibres were dried under nitrogen and cured at 70° C. for 3 hours. (Kang C K and Lee Y S, 2007). A schematic of the surface modification process is presented in FIG. 9.

Pre-Polymerization and Coating Material Preparation

A slurry was prepared by mixing the 1:10 3M-Scotch-weld DP240 epoxy adhesive and chloroform respectively. The slurry was mixed vigorously using vortex mixer (Ratek-VM1) and pre-polymerization was performed for 15 days at room temperature (22° C.). The pre-polymerization process was performed to increase the binding capacities of the fibre. The schematics of the pre-polymerization are shown in FIG. 1. After the pre-polymerization period, 0.4 gm/mL silica particles were mixed with the polymer and vortexed before coating the fibres.

N₂ Adsorption-Desorption Isotherm Analysis

BET (Brunauer-Emmett-Teller) surface area and pore volume were determined by nitrogen adsorption/desorption isotherms. The analysis was performed on a Micromeritics ASAP 2420 analyzer. Prior to the analysis the samples were degassed at 80° C. for 24 hours. To understand the pore-filling phenomenon, the BET analysis was performed on Day-1, 7 and 15. BET surface area was measured using the BET ( ) method in relative pressure range of

$$\frac{P}{{P}_{0}} 0.05 - 0.20$$

and total pore volume (Vt) was taken at P/Po=0.99. Data was analyzed using Sigma plot software (Systat software Inc, U.K). The analysis was performed using mesoporous hysteresis type-4.

The textural properties of the fibre showed an increasing surface area. We observed that control silica particles had a surface area of 179.9 m²/g⁻¹ with a wide pore size distribution whereas on Day-1 a 97.703 m²/g⁻¹ area was calculated. In comparison there was much higher surface area was observed after the 15-day pre-polymerization.

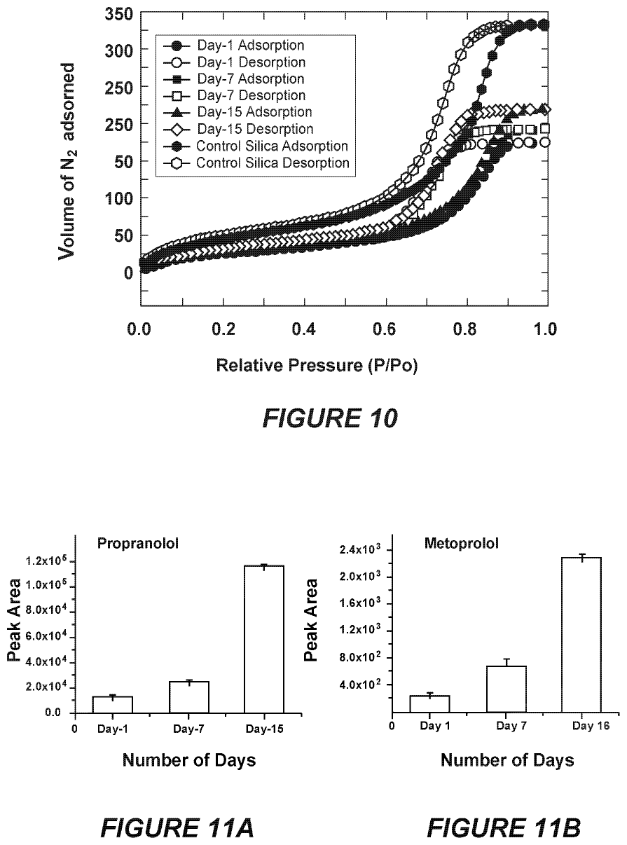

BET surface area was calculated form N₂ adsorption-desorption and the data are presented in Table 1 and FIG. 10.

TABLE 1
Textural properties of the pre-polymerized polymer
mixed with silica particles on different days

| Sample Name | BET SA (m²/g⁻¹) |
|---|---|
| Control | 179.918 |
| Day 1 | 97.703 |
| Day 7 | 102.621 |
| Day 15 | 115.361 |

HPLC-Binding Capacities

In order to validate the BET results from the pre-polymerized polymer, fibres were coated on different days and binding studies were performed using two β-blockers, metoprolol and propranolol. The analysis was performed using liquid chromatography and mass-spectrometry (Agilent). Maximum binding capacity was observed from the fibre which was coated on day-15. FIGS. 11(A) and (B) shows the binding capacity on different days. This analysis establishes the relationship with BET data analysis as similar trends were observed in binding capacity studies.

Coating Method and Optimization

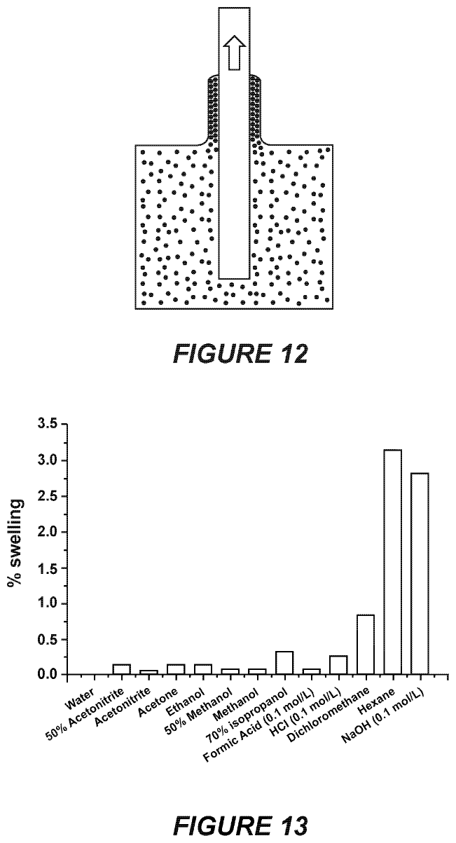

Dip-coating was performed using an in-house made computer software operated motor, a schematics of the dip coating procedure is shows in FIG. 12. Before performing the coating on the fibre, the slurry was mixed vigorously to achieve a homogenous suspension. Fibres were clamped on top of the motor and were dipped in the slurry for a length of 15 mm in height for four times (dip coating) to get the desired coating thickness (45 μm). After coating, fibres were cured at 75° C. for 60 minutes in an oven. In order to optimize the coating thickness on one fibre, multiple coatings were performed on each fibre and after every coating fibres were thermally polymerized. The coating thickness was measured microscopically.

Fibre Robustness Testing

Most of the fibre-based solvent extraction technologies have a problem of swelling and reduction in the extraction efficiency after exposing the fibres to various solvents. The breaking of the fibre coating mostly happens due to exposure to acidic or basic solutions, rigorous vortexing during the extraction process and extraction of the analytes from the stronger matrix. Swelling of the fibres was calculated as the ratio of the difference in coating thickness before and after solvent exposure to the original coating thickness multiplied by 100%. The coating thickness of a total of 39 fibres was measured optically and then all were exposed to the water, acetonitrile/water (1:1), acetonitrile, acetone, ethanol, methanol/water (1:1), methanol, 70% isopropanol, formic acid (0.1 mol/L), hydrochloric acid (0.1 mol/L), dichloromethane, sodium hydroxide (NaOH) and hexane for 15 minutes in a set of 10 each. All the experiments were performed in triplicate.

Table 2 and FIG. 13 show the results of the swelling % of the coating before and after exposure of the fibres to various solvents. The results show that no swelling was observed except exposure to the hexane and sodium hydroxide (NaOH) where the maximum swelling observed was 3.1% and 2.80%, respectively. There was no visible coating breakage, and no silica particles fallings were observed in the solvent vials after the exposure.

TABLE 2
The effect of the solvent on coating thickness
of biocompatible SiFT fibres

| Solvent | Swelling (%) |
|---|---|
| Water | 0.00 |
| Acetonitrile/Water (1:1 v/v) | 0.01 |
| Acetonitrile | 0.04 |
| Acetone | 0.12 |
| Ethanol | 0.13 |
| Methanol/Water (1:1 v/v) | 0.06 |
| Methanol | 0.28 |
| 70% Isopropanol | 0.309 |
| Formic Acid (0.1 mol/L) | 0.09 |
| Hydrochloric Acid (0.1 mol/L) | 0.26 |
| Dichloromethane | 0.82 |
| NaOH | 2.80 |
| Hexane | 3.14 |

Multiple Coatings on Fibres

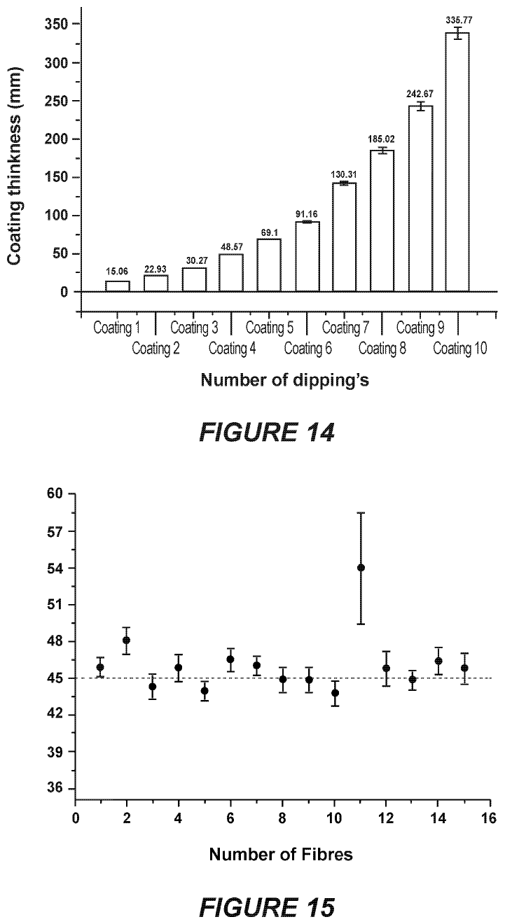

After performing multiple coatings on one fibre it was found that the required coating thickness (45 μm) was achieved after four dip coatings on the same fibre. Initial coatings had some voids on the surface whereas after two coatings the fibre surface was observed to be covered entirely with silica particles along with polymers and homogenous distribution throughout the surface was observed under the microscope. It was observed that a maximum of ten coatings can be performed and the coatings were very robust even after performing ten coatings on the fibre. However, it was also observed that the fibre could not hold any further coating after ten coatings. The maximum coating thickness achieved was 335 μm. FIG. 14 shows the multiple coatings on the fibres. The coatings were measured microscopically, and the thickness was observed a total of the nine spots in each fibre.

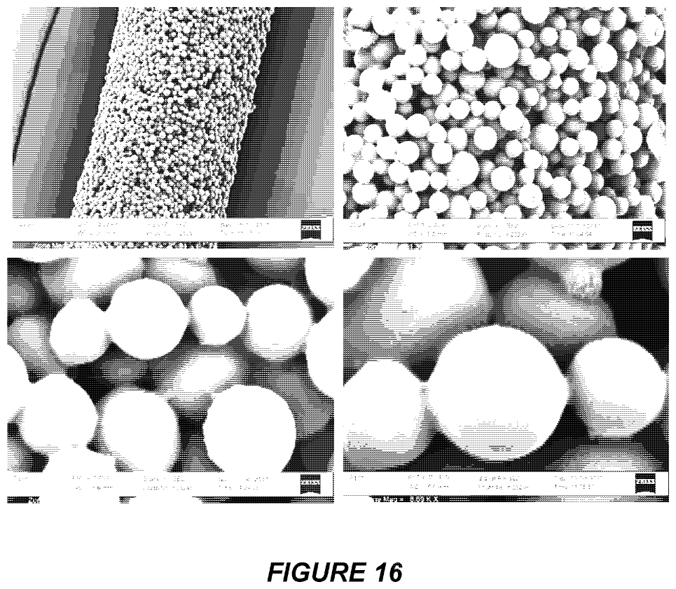

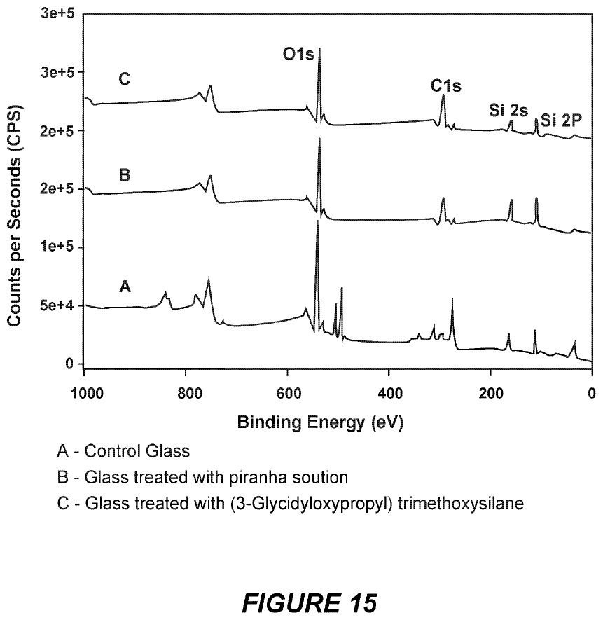

The coating thickness efficacy was also checked by measuring the coating thickness on 15 different fibres. It was found that the motor is very effective and controlled in terms of coating thickness on different fibres. Upon coating on 15 different fibres it was found that after four coatings a coating thickness of 45 (±) μm was attained. A total RSD (%) was 5.5% on 15 different fibres. The results of coating thickness observations are shown in FIG. 15.

Topological Characterization

Scanning Electron Microscopy Analysis

Surface morphology was assed using a Zeiss Merlin scanning electron microscope (Merlin, Carl Zeiss Co., Oberkochen, Germany) used at an operating voltage of 2 kV. The SEM images of the coated fibre demonstrate that the particles are completely covered with the polymer and silica particle distribution is homogenous throughout the coating FIG. 16 shows the SEM images of the coated fibre.

X-ray Photoelectron Spectroscopy (XPS)

X-ray photoelectron spectroscopy of the control, piranha treated, and silane treated substrate was carried out using a SPECS SAGE XPS system with a Phoibos 150 hemispherical analyzer at a takeoff angle of 90 and MCD-9 detector. XPS spectra was recorded from 0 to 1000 eV at a pass energy of 100 eV with the energy steps of 0.5 eV to determine the elements available on the differently treated glass substrates. Wide scan spectra were recorded for selected peaks using 0.1 eV energy steps at a pass energy of 20 eV. Spectra recorded for both silane coated, and control fibre were corrected by setting the aliphatic carbon peak by following the methodology of Beamson G and Briggs D., 1992. All the recorded spectra were analyzed using CASAXPS (Neal Fairley, U.K.).

Glass Substrates

The surface chemistry of the glass substrate was analyzed using XPS, the survey spectrum is displayed in FIG. 17 and in Table 3. The XPS analysis was performed on control, piranha treated, and silane treated fibres. Questioning the absence of silanol limiting the reaction, prior to silanization substrate hydroxylation was performed with the piranha solution. After the piranha treatment the surface shows less amount of carbon whereas silane treatment significantly increases the amount the carbon on the surface. These results show that treatment with the silane was found on the surface that lead to the robust binding of the polymeric material.

TABLE 3
XPS elemental composition on all three
different steps of modifications

| Substrate modification | O (%) | C (%) | Si (%) |
|---|---|---|---|
| Control | 52.030 | 16.264 | 31.706 |
| Piranha Treated | 51.361 | 15.634 | 33.055 |
| GPTS Treated | 40.397 | 36.374 | 23.229 |

Biocompatibility Testing

Preparation of a successful biocompatible coating for fibres shows a great capability of towards developing a device that can be used for biomedical, pharmaceuticals and forensic applications. For these applications, fibre-based device coatings need to be robust, thin and unbreakable. Resazurin assay for the cell-viability is widely used to evaluate the biocompatibility of polymeric materials. The evaluation was performed by measuring the reduction of resazurin to resorufin using spectrophotometers which happens due to transference of electron from NADPH+H⁺ to resazurin (Borra R C et al., 2009). Human foreskin fibroblasts HFF-1 (ATCC) were cultured in the DMEM (Dulbecco's modified Eagle's medium) (Life technologies, Victoria, Australia) supplemented with the 10% fetal calf serum (FCS) (AusGenex, Australia), 1% penicillin/streptomycin (Sigma-Aldrich) at 37° C. in the humidified 95% air and 5% CO₂ incubator. The media was changed every 3 days after observing 90% confluency of cells in the culture flasks, cells were washed using phosphate buffer saline (PBS) and detached using the 0.25% (w/V) trypsin solution (Sigma-Aldrich) in PBS. For this test, 4.5×10⁴ cells mL^{31 1} were seeded in a 6 well plate with 3 distinct groups (Control, uncoated fibre and coated fibre) in triplicates manner. All the experiments were performed between the passage number 9 to 13.

After allowing the cells to adhere to the surface for 8 hours, fibres placed in the nunc inserts were kept in the well. Cells were observed under a fluorescence microscope to check the cell structure every day. Prior exposing the fibres to the cells, all the fibres were sterilized in ethanol for 30 minutes followed by 45 minutes of ultra-violet (UV) exposure under controlled conditions. Cell-viability was quantified by resazurin assay at 24, 48 and 72 hours of incubation using 10% resazurin (Sigma-Aldrich). 500 μL Resazurin was added to each well and plates were incubated for 2 hours in CO₂ incubator. After the incubation period 100 μL of cell suspension was transferred to the 96 well plate and fluorescence was observed at 530ex/590em nm using a FLOUstar Optima plate reader (BMG LabTech Pty. Ltd, Victoria, Australia). All the statistical analysis was performed on GraphPad Prism software operated at windows 10.

Biocompatibility Evaluation

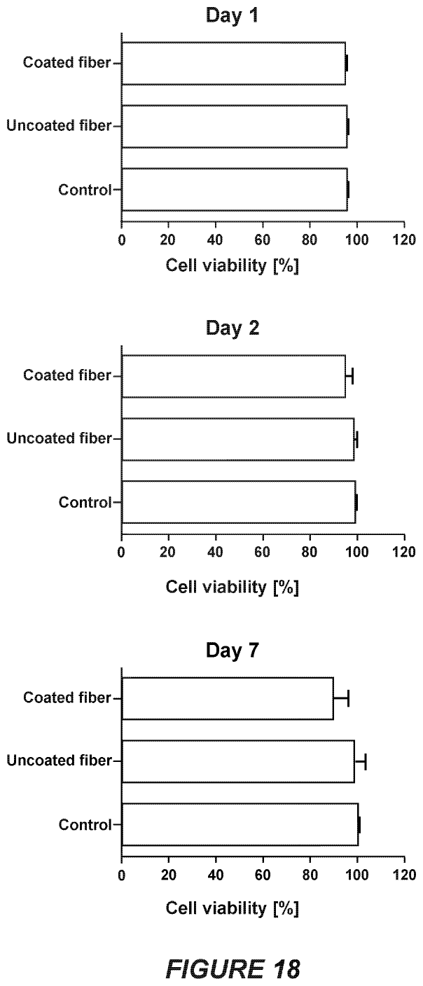

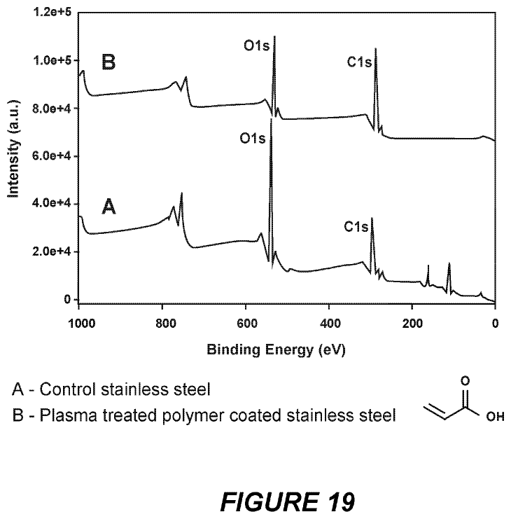

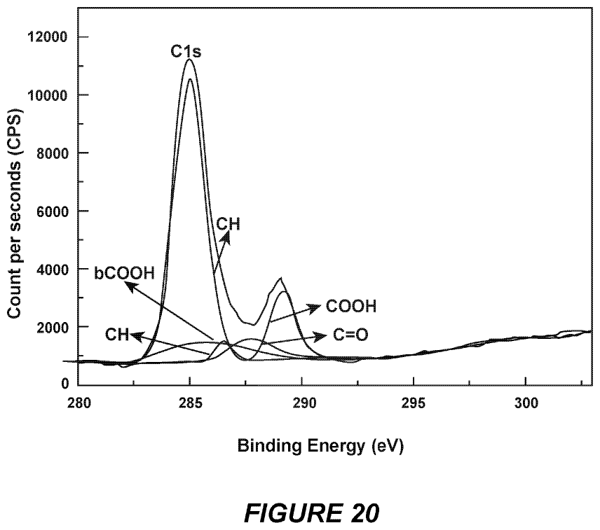

To test the cytotoxic potential of the coated fibres the resazurin assay was used to measure the cell viability after the exposure of the fibres to the primary HFF-1. To evaluate the biocompatible nature of the coated fibre primary human foreskin fibroblast cells were used instead of secondary cell lines. Fibroblast cells are an establish model to check the biocompatibility of the coated fibres. FIG. 18 shows the metabolic activity on different days after exposing the fibres to the cells. The results were compared with control cells. There was no statistically significant difference between the control cells, uncoated fibres and coated fibres (p<0.005). It was observed that there was no cell death (%) on day-1 or day-2 and the coated fibre had <10% cell death on day-7, which is an indication of over-confluent of the cells within the wells and lead to the leaching of the cells. These results suggest that coated fibres with silica particles and polymer had no adverse effect on the primary cells.

Surface Modification of Stainless Steel Substrates

A stainless-steel fibre (80 mm in length) was cleaned with dimethylformide (DMF) for 5 minutes in an ultrasonic bath, followed by cleaning with acetone, methanol and ultrapure water for 5 minutes each respectively. Fibres were dried under nitrogen and kept closed in a vial for further treatments. Plasma polymerization treatment was carried out in a custom made (high frequency, 13.56 MHz) plasma polymerization system and the power was supplied using an amplifier and matching unit of a coaxial power system (Coaxial Power Systems Ltd, Eastbourne, United Kingdom). Plasma polymer precursor, acrylic acid (AA) was purchased from the Sigma-Aldrich. At first, the plasma reactor chamber was evacuated using a rotary pump to a base pressure of below 1×10⁻⁴ mbar to remove all other atmospheric gases and impurities inside the chamber. Acrylic acid precursor was introduced to the chamber via a needle valve (Chell, U.K.) using precursor flow rate of 4 (cm³/min). The 50-watt (W) air plasma was run three times for 10 minutes each and fibres were rotated every time to make sure the coating of the plasma was even on the surface and covered the fibres. At the end of the process, the power was reduced to 5 watts for 20 minutes to deactivate the free radicals (Michelmore A et al., 2014, Kirby G T et al., 2017).

X-Ray Photoelectron Spectroscopy (XPS) of Stainless Steel Fibres

XPS of the plasma coated stainless steel fibres was carried out using a SPECS SAGE XPS system with a Phoibos 150 hemispherical analyzer at a takeoff angle of 90 and MCD-9 detector. XP survey spectra were recorded from 0 to 1000 eV at a pass energy of 100 eV with the energy steps of 0.5 eV to determine the elements available on the plasma coated and control stainless steel fibres. Wide scan spectra were recorded for selected peaks using 0.1 eV energy steps at a pass energy of 20 eV. Spectra recorded for both plasma coated and control fibres were corrected by setting the aliphatic carbon peak by following the methodology of Beamson G and Briggs D., 1992. All the recorded spectra were analyzed using CASAXPS (Neal Fairley, U.K.).

The content of the surface analysis of control steel fibre and acidic plasma coated fibres are listed in Table 4. The C1s core level spectra of the acidic plasma polymers were peak fitted using 70% Lorentian/30% Gaussian peak shapes with full-width at half-maxima (fwhm) between 1.6 and 1.9. There was significant amount of oxygen and carbon was present on the surface of the fibre. XPS spectra also shows the increase of the carbon (%) from control substrate to the plasma treated stainless steel substrate.

TABLE 4
Content of the surface analysis of control
steel fibre and acidic plasma coated fibres

| Name | At % |
|---|---|
| COOH | 12.86% |
| bCOOH | 12.815% |
| CH | 64.928% |
| C═O | 6.471% |
| C—OH | 2.959% |

SEM Analysis of Stainless Steel Fibres

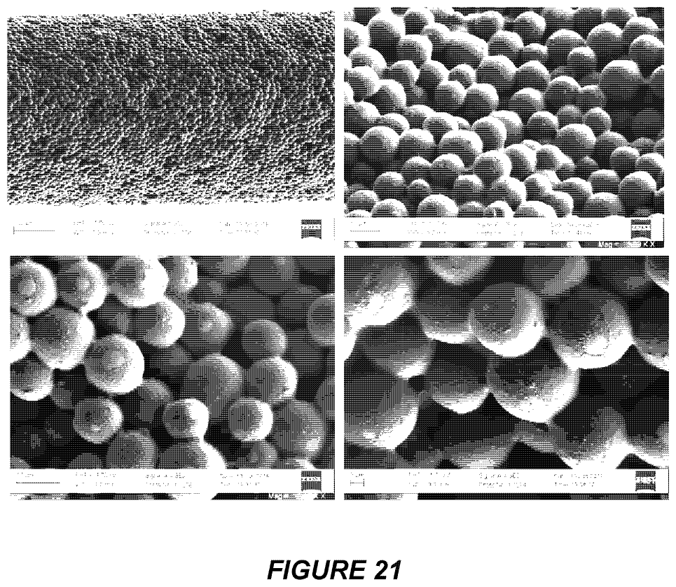

The morphology of the coating on the stainless-steel fiber was observed under high-resolution SEM show in the FIG. 21. The SEM images analysis shows the mixture of silica particles and polymer distribution was coated homogenously on the surface of the stainless fiber. It was found that silica particles are evenly distributed throughout the surface with spherical morphologies and polymer attachment can be seen on the silica particles.

### Example 5

### Substrate with Protein Resistant Coating

SiFT fibres/rods were modified with C18 particles with 120 Å pores. A 1 mg/ml solution of 2-nitroaniline in water was added to water and foetal calf serum in ratios 1:1 and 1:5 resulting in concentrations of 0.5 and 0.1 mg/ml respectively. A glass rod coated with 4 mm of coating was used for the binding studies. The rod was submerged in the solutions for 5 minutes and the bound analyte was eluted with 1 ml of methanol. 20 μl of the elution solution was injected into a HPLC.

When the sample is dissolved in serum, there are multiple sample components competing for the binding sites, hence, the binding of 2-nitroaniline is reduced. The effect is more pronounced when the analyte of interest is in a lower concentration.

In order to suppress protein binding the fibres were coated with a second layer of dextran. Dextran is a hydrophilic polysugar and only shows minimal interactions with proteins. The dextran chosen had a molecular weight of 450,000 to 600,000 with a Stokes radius of 150 Å. Therefore the dextran molecules cannot penetrate the pores but form a hydrophilic barrier to prevent proteins and other large molecules to come in contact with the C18 particles. In brief, an aqueous solution of dextran was precipitated onto the coating and crosslinked with 1,4-butanediol diglycidylether. The dextran coating was performed on three rods and the binding properties were evaluated as before however only with the 0.1 mg/ml concentration. The results are shown in Table 5.

TABLE 5
Binding properties of dextran coated substrates

| | dextran coated | | |
|---|---|---|---|
| | water | serum | % |
| Rod #1 | 108657 | 68132 | 63 |
| Rod #2 | 109454 | 105439 | 96 |
| Rod #3 | 121327 | 93960 | 77 |

With the untreated rods a 42% relative binding was achieved when the sample was in serum. After the coating an increase the relative binding to 63, 77 and 96% was observed. Thus, a complex matrix can reduce the binding capacity of the substrates for a target analyte and by suppressing the proteins in serum to compete for the binding sites the relative binding capacity for 2-NA can be increased significantly.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgment of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

### REFERENCES

Michelmore A, Whittle J D, Short R D, Boswell R W, Charles C. An Experimental and Analytical Study of an Asymmetric Capacitively Coupled Plasma Used for Plasma Polymerization. Plasma Processes Polym. 2014 11, 833.

Kirby G T, Mills S J, Vandenpoel L, Pinxteren J, Ting A, Short R D, Cowin A J, Michelmore A, Smith L E. Development of Advanced Dressings for the Delivery of Progenitor Cells. ACS Appl Mater Interfaces. 2017 Feb. 1; 9(4):3445-3454.

Beamson, G.; Briggs, D. High Resolution XPS of Organic Polymers: The Scienta ESCA300 Database; Wiley: Chichester, U.K., 1992.

Borra R C, Lotufo M A, Gagioti S M, Barros Fde M, Andrade P M. A simple method to measure cell viability in proliferation and cytotoxicity assays. Braz Oral Res. 2009 July-September; 23(3):255-62.

1. A solid phase microextraction substrate having a sorbent coating on at least part of a surface thereof, the coating being adapted for extracting at least one analyte component from a fluid matrix, the coating comprising sorbent particles in a polymeric adhesive matrix, and wherein a majority of pores in each sorbent particle in the coating do not contain substantially any of the polymeric adhesive matrices.

2-7. (canceled)

8. The solid phase microextraction substrate of claim 1, wherein the polymeric adhesive matrix is a polyamine epoxy.

9. The solid phase microextraction substrate of claim 8, wherein the epoxy component of the polyamine epoxy is an epoxy resin having at least two epoxy groups.

10. The solid phase microextraction substrate of claim 8, wherein the polyamine component of the polyamine epoxy is an aliphatic polyamine or a cycloaliphatic polyamine curing agent.

11. The solid phase microextraction substrate of claim 1, further comprising a biocompatible outer coating.

12. (canceled)

13. The solid phase microextraction substrate of claim 1, further comprising a hydrophilic outer coating.

14-16. (canceled)

17. A process for preparing a solid phase microextraction substrate having a sorbent coating on at least part of a surface thereof, the coating being adapted for extracting at least one analyte component from a fluid matrix, the process comprising:
forming a sorbent particle/adhesive precursor composition comprising a polymeric matrix adhesive precursor material and sorbent particles under conditions to substantially prevent ingress of the polymeric matrix adhesive precursor material into pores of the sorbent particles in the sorbent particle/adhesive precursor composition;
coating at least part of a substrate with the sorbent particle/adhesive precursor composition; and
polymerising the polymeric adhesive precursor material in the sorbent particle/adhesive precursor composition under conditions to form a sorbent coating comprising sorbent particles in a polymeric adhesive matrix.

18. The process of claim 17, wherein the conditions to substantially prevent ingress of the polymeric matrix adhesive precursor material into pores of the sorbent particles comprise blocking the pores of the sorbent particles with a pore blocking agent.

19. The process of claim 18, wherein the pore filling agent is selected from the group consisting of hexadecanol, paraffin waxes and long chain alcohols.

20. The process of claim 18, wherein the blocked pore sorbent particles in the polymeric adhesive matrix are treated to substantially remove the pore filling agent from the pores thereof to form the sorbent coating comprising sorbent particles in a polymeric adhesive matrix.

21. The process of claim 20, wherein the treatment comprises heating the sorbent particles after coating onto the substrate.

22. The process of claim 17, wherein the conditions to substantially prevent ingress of the polymeric matrix adhesive precursor material into pores of the sorbent particles comprise contacting the sorbent particles with adhesive matrix precursor polymers or pre-polymers that have a molecular size that is greater than a maximum pore size of the sorbent particles.

23. The process of claim 22, wherein at least part of a surface of a substrate is coated with the sorbent particle/adhesive precursor composition after which it is polymerised under conditions to form a sorbent coating comprising sorbent particles in a polymeric adhesive matrix.

24. The process of claim 22, wherein the adhesive matrix precursor polymers or pre-polymers are formed by starting polymerisation of the polymeric adhesive material and adding the sorbent particles to the reaction after polymerisation has started but before it is finished.

25. The process of claim 24, further comprising monitoring the state of polymerisation of the adhesive matrix precursor material.

26. The process of claim 25, wherein the state of polymerisation of the adhesive matrix precursor material is monitored by measuring the viscosity of the reaction mixture.

27. The process of claim 26, further comprising surface treating the substrate prior to it being coated with the sorbent particle/adhesive matrix precursor composition.

28. The process of claim 27, wherein the surface treatment comprises hydrolysis.

29-36. (canceled)

37. The process of claim 17, wherein the polymeric adhesive matrix is a polyamine epoxy.

38-46. (canceled)

47. Using the solid phase microextraction substrate of claim 1, A solid phase microextraction process.
